(19) United States
(12) Patent Application Publication
(10) Pub. No.: US 2025/0012752 A1
(43) Pub. Date: Jan. 9, 2025

(54) SOIL PARAMETER SENSOR SYSTEM

(71) Applicant: Soil In Formation, PBC, Saint Louis, MO (US)

(72) Inventors: Shalini Prasad, Allen, TX (US); Vikram Narayanan Dhamu, Dallas, TX (US)

(73) Assignee: Soil In Formation, PBC, Saint Louis, MO (US)

(21) Appl. No.: 18/711,138

(22) PCT Filed: Mar. 4, 2022
(86) PCT No.: PCT/US2022/018910

§ 371 Date: May 17, 2024

Related U.S. Application Data
(60) Provisional application No. 63/299,652, filed on Jan. 14, 2022.

(51) Int. Cl. G01N 27/30; G01N 33/24

(52) U.S. Cl. CPC G01N 27/30; G01N 33/246

(57) ABSTRACT

An in-situ, or interfacial, soil sensor is provided, which includes one or more electrodes. At least one of the one or more electrodes includes a room temperature ionic liquid (RTIL)-based or other functional chemical material-based film coating, where the electrode is to be brought into direct contact with a soil sample, and the soil sensor is functionalized to detect one or more characteristics of the soil sample and generate a signal corresponding to the one or more characteristics. The film coating enables sensing of a corresponding specific soil health attribute and can be used as one or more of an active element, support electrolyte, or binding element for the soil sensor.

### CROSS-REFERENCE TO RELATED APPLICATION

This application claims priority to U.S. Provisional Application 63/299,652 filed on Jan. 14, 2022, the content of which is incorporated herein in its entirety.

### BACKGROUND

Soil and water are vital components of the Earth's ecosystem, both playing a major role in maintaining the ecological balance of the planet and sustaining mankind. For instance, the correct use of resources such as water and fertilizers in agriculture application has enormous societal (e.g., health, economic, etc.) importance as well as importance at an environmental level. Governments, corporations, and non-profit groups are working to develop paradigms in which soil and water are protected, regenerated and used more intelligently for the benefit of man and nature. For instance, “regenerative farming” concepts and practices have been proposed and implemented for improving soil health, increasing nutrient levels in crops, improving water use efficiency and reversing climate change through sequestering carbon.

For farmers, the systematic recording of a wide range of information about their fields helps them to implement quality regenerative farming systems, with the aim of improving the information which the farmers rely on for planning and decision-making to increase productivity, land quality, land assets and to reverse losses or inefficiencies in their farms. Optimization of regenerative agriculture is also important at a macro societal level, by protecting crop-producing land, water availability, quality and safety, and reversing negative environmental impacts due to degenerative, inefficient or careless farming practices.

Traditionally, analytical information for soil health has been obtained by means of manually taking soil samples from various accessible zones to represent various soil regions. The samples are then transported to a laboratory to conduct tests and measurements on these samples to better understand the individual nutrient levels in these soil samples and to estimate the overall soil health and water use efficiency in a whole field, farm or in wider regions. Such traditional measurement systems are both inefficient, suboptimal, and expensive, limiting the utility and affordability to the end-user farmers, and thereby limiting the accessibility and momentum needed to implement such “regenerative farming” methods at scale, among other example disadvantages.

### BRIEF DESCRIPTION OF THE DRAWINGS

To provide a more complete understanding of the present disclosure and features and advantages thereof, reference is made to the following description, taken in conjunction with the accompanying figures, wherein like reference numerals represent like parts, in which:

FIG. 1 is a simplified block diagram illustrating an example soil sensor system deployed on a plot of land.

FIG. 2 illustrates example cations and anions capable of being used in the formulation of room temperature ionic liquids (RTIL) for application to example soil sensors.

FIGS. 3A-3C are diagrams illustrating principles of an example soil moisture sensor.

FIGS. 4A-4C are diagrams illustrating principles of an example soil volumetric density sensor.

FIGS. 5A-5C are diagrams illustrating principles of an example soil organic matter sensor.

FIGS. 6A-6C are diagrams illustrating principles of an example carbonous soil minerals sensor.

FIGS. 7A-7C are diagrams illustrating principles of an example soil pH sensor.

FIG. 8 is a simplified flow diagram illustrating a technique for using an example soil sensor.

Like reference numbers and designations in the various drawings indicate like elements.

### DETAILED DESCRIPTION

Room temperature ionic liquid (RTIL) is a unique chemical compound, which possesses excellent physical, chemical, and electrochemical properties, which enables such species to be utilized as a transducer for probing a complex matrix such as soil. The wide electrochemical window and elevated double layer capacitance of RTIL helps to gauge soil parameters which is helpful to understand soil state. Soil health and quality is a foundational measure of a functional, self-sustaining environment.

Standard techniques typically involve empirical approaches, extensive sample preparation which adds on to a temporal factor along with equipment for extraction and subsequently-analysis. This therefore stimulates the need for a testing methodology that is capable of rapid analysis in an in situ environment that can be applicable universally. In some implementations, a rapid electrochemical point probing mechanism may be provided that acts as a soil state evaluation platform via a 3-electrode sensor modified by a widely characterized-RTIL [BMIM] [BF4] interfacial transducer medium. Therefore, by looking at the rate of electrochemical activity and inherent soil dielectric changes driven by an RTIL electrode-soil interfacial layer, it is possible to decouple information on nutrient levels and availability in soils with potential for application towards temporal soil analysis.

Room Temperature Ionic Liquids (RTILs), a class of ionic liquid family, is a fascinating compound for its unique physico-chemical as well as electrochemical property and hence it is able to be utilized as an electrolyte system to allow electrochemical transduction. Basically, RTIL is a compound made of cation and anion that sit side-by-side similar to a zwitter-ion, making the total charge of the compound neutral. Along with ionic conductivity, RTILs also possess decent capacitance as well as the ability to diffuse gas molecules along its interface, among other example features. Features of RTIL compounds enable RTILs to be usefully deployed in electrochemical probing. For instance, properties of RTILs encompass high chemical and thermal stability, wide electrochemical window, and negligible vapor pressure, which makes them superior transducers compared to conventional nanoparticle-based transducers. The unique electrochemical property of RTILs allows analytes to diffuse, resulting in steady state diffusion current upon applying potential. Along with diffusion of charges across its interface, RTILs also possess high double layer capacitance, which enable RTILs to be deployed as impedimetric sensors.

In some implementations, electrodes of an improved in situ soil sensor may be coated with an RTIL to enable sensing of particular characteristics of soil. Such improved interfacial sensors may utilize RTIL as an active element to detect the presence and concentration of various chemical molecules within the soil. For instance, different combinations of cationic and anionic species allow the tuning of an RTIL coating to functionalize the RTIL as an active element of a soil sensor configured to detect a particular target molecule. In other example, RTIL may be utilized as a support electrolyte. For instance, an electrolytic contact between the sensor and the soil system may be bridged by the use of the RTIL film to modify the electrode system. RTIL may also be used as a binding element in an in situ, interfacial soil sensor. For instance, a non-RTIL compound may be applied as the active element in a particular soil sensor, but this compound may not, by itself, possess the characteristics to enable its deployment in an in-situ soil sensor. For instance, the compound may react or otherwise respond poorly to soil conditions (e.g., dissolve off the electrode in the presence of moisture in the soil). An electroactive RTIL film may be provided over the active element layer to stabilize and modulate the active element, while allowing it (and the sensor itself) to function as intended.

Soil is a fundamental core element of the environment we live in that directly impacts the growth of plants, crops and other vegetation in addition to having a relationship with other parts of the ecosystem including water and air. There is a major requirement for information related to the physical, chemical and biological components of soil and thereby its vital association with soil health. Among these groups of parameters, it can be said that studying chemical profiles of the soil can be used to create a soil health index with a high degree of reliability in an in-situ manner. This is possible, because soil chemistry can be derived using in-field probes that have a significant correlation and are also a function of the physical and biological parameters as well as activity in the soil matrix. Commercially, soil quality determines crop yields and cost of farmland, wherein it is essential to have thorough dynamic and in-situ information about soil parameters that is synchronized in terms of geological location (space) and period (time) due to variations associated with environmental and land use changes. Implementation of dynamic, in-situ soil sensors would therefore be of utmost benefit to characterize multiple soil parameters related to soil health from a local as well as global environmental-impact standpoint.

Soil health/quality is defined by its capacity to function as a sustainable ecosystem that supports plants, animals, and humans alike. Typically, soil health includes three types of soil characteristics: biological, physical, and chemical. Although sometimes used interchangeably, soil quality, from a practical sense, refers to soil chemical and physical properties. For instance, soil health assessment in large part is determined by the nutrient levels in soil. Hence, assessment of soil health parameters in an on-farm setting, facilitates quantification and recording of the soil's inherent physio-chemical and biochemical characteristics. Sufficient levels of soil nutrients are required for sustainable agricultural practices that typically increase the health of the agricultural ecosystem and boost crop yields, pasture growth, etc. Currently, soil sampling and evaluation methods involve intrusive approaches to collect and then subsequently test the soil samples in a laboratory environment that differs from the point of collection. Among these, the Combustion method is one of the most widely sought-after techniques to look at soil anatomy. Even with the upcoming research breakthroughs in non-destructive approaches like spectroscopy and tomography-based models, there is a barrier in-terms of accuracy of the methods, especially at soil depths below 5-10 cm, equipment complexity and availability as well as high costs and high logistical overhead, among other issues.

There is a wide number of primary, secondary and micronutrients that are required by plants for growth in addition to the dire need for the soil organic matter pool. An important metric that needs to be considered here is that the rate of nutrient release for uptake by the crops or plants is affected by the availability of the various nutritional sources and other soil parameters. There are a number of electrochemically active and redox substances present in soil that exist in reduced state under ideal conditions (submerged) that contribute to electrochemical activity and in turn have a proportional effect on the soil quality.

Some soil properties often used to evaluate soil physical properties are bulk density, infiltration parameters, water holding capacity, and soil texture; on the other hand, parameters used for chemical evaluation typically include soil pH, plant available nutrients, soil nitrate, reactive carbon, soil organic matter, and electrical conductivity. Biological properties of soil systems include the diversity and quantity of soil organisms (soil food web), total organic carbon, soil respiration, and soil enzymatic activity. Overall, each of these individual parameters can be matched to provide information about the soil state which in turn is the end objective.

Improved sensors may be provided, which leverage soil electrochemistry to correlate soil health in terms of understandable electrochemical signals. Such sensors may be implemented as integrated and miniaturized platforms along with reliable data output. For instance, a sensor device may be implemented as an on-chip in-situ diagnostic platform for continuously monitoring active parameters inside the dynamic soil ecosystem. Using such sensors, data sets may be collected and processed to identify correlations between electrochemical activity and the presence of active substances that contribute to the soil nutrient cycle. Indeed, such improved in-situ soil sensors (e.g., utilizing RTIL and other functional materials coatings) may permit soil health to be assessed and monitored at an interfacial level using a probe system. Accordingly, the soil matrix may be characterized using the resulting data in order to provide information in terms of various physio-chemical phenomena occurring at the electrode interface. Subsequently this information can be used to correlate that to useful data which helps to understand soil fertility and bioavailability of nutrients for plants and other vegetation at the field level, among other example insights.

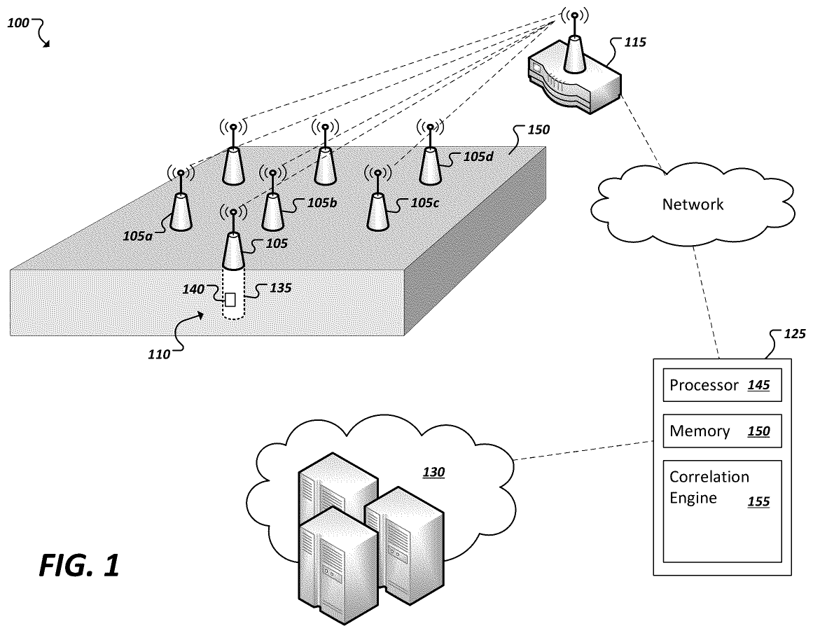

Turning to FIG. 1, a simplified block diagram 100 is shown illustrating an example soil sensing system implemented using one or a collection of computing and/or sensor devices. In one example system, a set of sensors (e.g., 105, 105a-d)—at least some of which include RTIL films—may be deployed in an agricultural plot 150 to test various areas, or samples (e.g., 110), of soil under various electrochemical modalities to thereby visualize the composite soil chemistry profile of the plot via a point measurement from different characterization perspectives. Such different perspectives may be collected utilizing a collection of sensors (e.g., 105, 105a-d), which includes sensors dispersed in various areas of the plot and/or different types of sensors (e.g., measuring the same or varied portions of the plot), as well as collecting measurements from the sensors on a rolling or continuous basis so as to survey the development of the soil's health attributes over time. Thus, improved data and resulting insights may be derived in such on-field applications due to the sensor devices capturing the dynamic behavior of soil through sensor readings in a range of temporal and spatial settings. Integration of these measurements from spread-out temporal and spatial points may be used by the system to compute a holistic soil profile for the corresponding region, among other example applications and potential benefits.

In some implementations, supplemental or cooperating computing systems may be provided to communicate with and consume data generated by the collection of sensors (e.g., 105, 105a-d). In one example, a gateway device or other I/O device (e.g., 115) may be utilized to collect signals and other data generated by the sensor devices (e.g., 105, 105a-d) and collect, aggregate, filter, and/or sort the data for consumption by other computing systems and logic. For instance, a computing system (e.g., 125) may be provided with computational logic to determine correlations between the readings of the sensors (e.g., 105, 105a-d) and corresponding soil attributes, which the sensors are configured to measure. For instance, a sensor (e.g., 105) may include one or more electrodes (e.g., 140), which are to contact soil (e.g., 110) and measure electrochemical characteristics of the soil. The electrode(s) 140, in some implementations, may be coated in an RTILfilm to enable the electrode to function appropriately within the sensor 105 to enable the sensor to detect certain soil characteristics. For instance, the sensor 105 may generate signals based on these measured electrochemical characteristics. The signals, by themselves, may not directly indicate the level of certain soil health attributes, but through analysis by a correlation engine 155 (e.g., implemented in software and/or hardware of a computing system (e.g., 125)), correlations between certain electrochemical characteristic measurements and corresponding levels of one or more soil health attributes may be determined. While FIG. 1 shows that correlation engine 155 may be executed by a processor 145 and stored in memory 150 of a computing system remote from the sensors (e.g., 105, 105a-d), in some implementations, the hardware and logic of system 125 may be integrated on the sensors themselves to allow this translation between electrochemical readings and various soil health attribute measurements to be determined locally. In some implementations, soil health attribute results determined by a correlation engine 155 may be shared with other computing systems for further storage and/or processing, such as a cloud-based soil-health analysis system (e.g., 130) among other example implementations.

In some implementations, a method and system are provided for tracking soil state by assessing soil nutrient levels in the matrix through characterizing the electroactive elemental changes associated with an RTIL modified electrode of a sensor device as a function of nutrients spiked in this soil construct. For this purpose, both faradaic and non-faradaic electrochemical approaches may be utilized to examine the electrochemical changes in signal due to the presence of various redox and electroactive species in the system. This work is believed to be novel and thereby demonstrates proof of feasibility towards visualizing the distinctions between soil structure, soil nutrient content and soil nutrient uptake variables associated with the soil system under test. This work aims to help determine whether any methods to enrich soil through changes in agricultural practices or addition of nutrients or amendments causes changes to the electrochemical signal. Based on our assessment of the field, this work is the first attempt at evaluating the electrochemical probing process as a means towards assessing soil health through soil structure and soil nutrient state. This has been achieved through characterizing the charge transfer (faradaic) indicative of ion movement or electrolytic behavior vs capacitive modulation (non-faradaic) that represents innate soil state dynamics leading the overall results attributing to soil state. A number of electrochemical modalities (DC as well as AC techniques) have been utilized in conjunction to probe soil properties in a multi-dimensional manner and hence be used to evaluate a comprehensive soil anatomy profile.

As noted above, a variety of RTIL compounds may be developed through the combination of respective cations and anions. Different RTILs may exhibit characteristics, which enable their application in various soil sensor implementations. Table 1 illustrates some of the example RTIL compounds that may be used in in-situ soil sensor implementations, such as discussed herein:

TABLE 1
Example RTIL Compounds

| lonic Liquid (IL) | Compound Name |
|---|---|
| [BMIm]PF6 | 1-Butyl-3-methylimidazolium |
| | hexafluorophosphate |
| [BMIm]BF4 | 1-Butyl-3-methylimidazolium |
| | tetrafluoroborate |
| [BMP]Tf2N/NTf2 | 1-Butyl-1-methylpyrrolidinium |
| | bis(trifluoromethylsulfonyl)imide |
| [BMIm]Tf2N/NTf2 | 1-Butyl-3-methylimidazolium |
| | bis(trifluoromethylsulfonyl)imide |
| [BMMIm]Tf2N/NTf2 | 1-Butyl-2,3-dimethylimidazolium |
| | bis(trifluoromethylsulfonyl)imide |
| [BMIm]TfO | 1-Butyl-3-methylimidazolium |
| | trifluoromethanesulfonate |
| [EMIm]BF4 | 1-Ethyl-3-methylimidazoliumtetrafluoroborate |
| [OMIm]Tf2N/NTf2 | 1-Octyl-3-methylimidazolium |
| | bis(trifluoromethylsulfonyl)imide |
| [PMP] | 1-Propyl-1-methylpyrrolidinium |
| [EMIm]PF6 | 1-Ethyl-3-methylimidazolium |
| | hexafluorophosphate |
| [EMIm]Cl | 1-Ethyl-3-methylimidazolium chloride |

Among several electrochemically useful RTILs, 1-butyl-3-methyl imidazolium tetra fluoroborate [BMIM] [BF4] possesses high ionic conductivity, wide electrochemical window and superior double layer capacitance, among other properties. In some implementations, RTIL may be used as an electrochemical transducer to probe a complex matrix like soil. In some implementations, 1-butyl-3-methylimidazolium tetrafluoroborate [BMIM][BF4] may be employed as the RTIL electrochemical transducer. The density of RTIL is higher than water and the vapor pressure is negligible and hence it may be advantageously used for many important analytical applications. This low volatility and exceptional thermal stability (e.g., up to 400 K) compared to other organic solvents, makes RTIL an excellent tool within electrochemical applications.

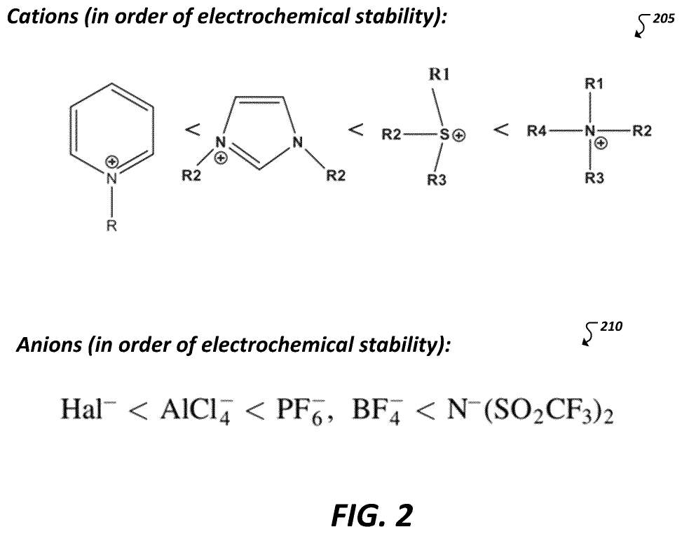

There exists a complex relationship between viscosity and ionic conductance that is understood by Walden's rule which suggests that mostly long alkyl chain hydrocarbon cation based RTIL possesses lower conductivity based on restrained charge distribution. FIG. 2 illustrates the relative electrochemical stability of a subset of cations 205 and anions 210 that may be incorporated as the cations and anions (respectively) of an RTIL. Indeed, any combination of such cation and anion may result in different RTILs exhibiting a similarly diverse array of physical chemical and electrochemical properties. Among several possible RTILs having the above combination present either in cation or anion, [BMIM] [BF4] is considered one of the prime candidates to be used as a transducer for electrochemical application. It has an ionic conductivity of 3.15 mS cm-1 whereas it possesses a wide electrochemical window of 4.9 V. This makes the RTIL very effective compared to other combinations.

To understand the electrochemical properties of an example RTIL transducer system, electrochemical impedance characterization was performed via non-faradaic EIS. Kosmotropic fluorinated anion-BF4 in surrounding conjunction with cation BMIM, forms the EDL structure with great capacity towards electrostatic and hydrogen bonding 32-34. For instance, structures with dual polarity, like [BMIM][BF4], can interact with species having+ve/−ve charges via electrostatic, —H bonding, non-covalent interaction. This zwitter-ionic structure thereby also increases the capacitance of the system due to its inherent electrochemical activity. It also enables novel profiling applications due to their calibration towards various electroactive and passive (bulk) moieties. Hence, a complex system like soil with various functional groups can be reliably characterized using modulation or capacitance changes in this double layer construct.

In some implementations, a mechanism of an RTIL sensor can be attributed to the contact surface between the RTIL and the electrode causing a formation of an electrical double layer. In some implementations, the RTIL can be a BMIM BF4 RTIL or other tetra fluro burate anion-based RTIL, among other examples. This electrical double layer (EDL) is prone to modification due to various effects at the electrode-electrolyte interface including adsorption of ions, steric effects, and electrolyte modification due to charge surface addition to the interface. This thereby causes an alteration to the EDL influencing its structure and thickness. This can be denoted by the mapping of the interfacial properties and translating the modifications of the EDL to the addition of nutrients or charged species (bulk). In some implementations, electrochemical impedance spectroscopy and subsequent equivalent circuit fitting may be utilized to determine what in the EDL is driving the change as a function of soil species addition.

In the non-faradaic analysis, it is observed that the application of an AC bias perturbs the double layer causing a diffusion effect that is representative in Table 2. This implies that the species in the soil matrix are innately disrupted causing a movement within the bulk electrolyte. This diffusion metric varies as a function of the species in the holistic soil system. Similarly, in the presence of a tag in the faradaic analysis, the electrical field bias causes a polarization and affects the EDL causing a pseudo-capacitive change along with Rct variation due to the redox label presence (Table 3). This also indicates that the redox tag amplified/mediated signal is dependent on the electroactive nutrients present in the soil sample.

TABLE 2
Parameter values extracted from fitted
EIS model for non-faradaic analysis

| Sample | Rs | Wo-R | Wo-T | Wo-P |
|---|---|---|---|---|
| Soil Baseline | 525 | 1387 | 305.77E−5 | 0.49991 |
| 0.4% amendment | 48.25 | 60.86 | 10.188E−5 | 0.47057 |
| 4% amendment | 52.13 | 49.32 | 7.9349E−5 | 0.46059 |

TABLE 3
Data values extracted from fitted model for faradaic EIS analysis

| Sample | Rs | CPE-T | CPE-P | Rct |
|---|---|---|---|---|
| Soil Baseline | 32.84 | 1.0832E−5 | 0.83823 | 427.5 |
| 0.4% amendment | 26.85 | 7.6714E−6 | 0.86959 | 637.7 |
| 4% amendment | 79.76 | 1.0048E−6 | 0.85936 | 1183 |

According to various embodiments, the sensor devices described herein may be adapted such that analysis of a species of interest may be conducted using the interfacial soil sensor devices, in one embodiment, in the devices described herein, or in another embodiment, downstream of the devices described herein, for example, in a separate server (e.g., 125, 130) coupled to the device (e.g., 105). It is to be understood that the devices described herein may be useful in various analytical systems, including bioanalysis microsystems. Although the biosensor system has been described with respect to particular devices and methods, it will be understood that various changes and modifications can be made without departing from the scope of the embodiments.

The RTIL is studied for this type of analysis and tentative system due to its ability to be strongly oriented at the double layer with a superior local ion density. This translates to increased transduction capability. Additionally, the use of RTIL and formation of RTIL electrode EDL is sought after because of its innate polarizable nature, complex zwitter-ionic structure that can prove to be sensitive towards a wide set of physical and chemical interactions.

More generally, soil nutritional state composition may be sensed and analyzed using an RTIL modified electrode system in a faradaic as well as non-faradaic fashion via multi-modal electrochemistry. For instance, a set of sensors (e.g., 105, 105a-105d, etc.) may be developed to simultaneously detect various soil parameters over time. These parameters may be used as surrogate measures towards assessing soil health. The sensing methodology is based on an electrochemical analytical approach with the potential for in-situ soil applicability. The mechanism explained in this document explores the utility of an interfacial electroanalytical approach that studies the following soil parameters by probing the soil sample in a point-level manner based on the physical contact made by the sensor. Some of these sensors may utilize RTIL-based sensors to detect these parameters.

For instance, in one implementation, a sensor device and system may be provided, which is functionalized to detect two or more of the following parameters within a soil sample (e.g., in which the sensor is deployed): soil hydration state, soil volumetric density, soil organic matter, carbonous soil minerals, and soil pH. At least one of the parameters, in one example, may be sensed using RTIL-based sensing.

Traditional soil health monitoring in the near past has been highly qualitative and speculative with more recent advancements still trying to fill the void of determining a holistic soil profile through surrogate measurements. Testing methods to perform soil profiling often differ from one lab to another and hence results are not universally compatible. Additionally, these normalized techniques often involve empirical approaches, extensive sample preparation which adds on to a temporal factor along with equipment for extraction and subsequently-analysis. Among these, combustion-based analysis is one of the most widely sought-after techniques to look at soil anatomy. Even with the upcoming research breakthroughs in non-destructive approaches like spectroscopy and tomography-based models, there is a barrier in-terms of accuracy, especially at depths of more than 10 cm in-field, equipment complexity and availability as well as high costs and high logistical overhead.

In an improved system, a set of electrochemical sensors may be deployed for dynamic in-soil use, the sensors functionalized with surface treatments to enable physico-chemical interactions or capture target analytes in soil when an input electrical signal is applied, and then the occurring interaction is transduced to an equivalent electrical output signal that varies from the sensor baseline. This specific change in output is recorded (e.g., locally on the sensor or by a cooperating device in communication with the sensor) and labeled as target levels of the analyte under test. Analytes may be selected to functionalize the respective sensor to detect each of the following parameters: soil hydration state, soil volumetric density, soil organic matter, carbonous soil minerals, and soil pH, among other examples. In some implementations, a single sensor device may be functionalized to sense two or more of these parameters.

In some implementations, signal capture of the enhanced sensor system is tracked based on the following modes:
Potential modulation due to ionic systems present in the soil system.
Charge modulation due to organic moieties present in the soil system.
Current based changes due to compositional changes in the soil system.
Dielectric (Diffuse double layer) based model to track resistive and capacitive changes in signal as a function of bulk/physical soil composition.
Charges movement from ions present in soil system towards functionalized sensor that captures this as a potential change

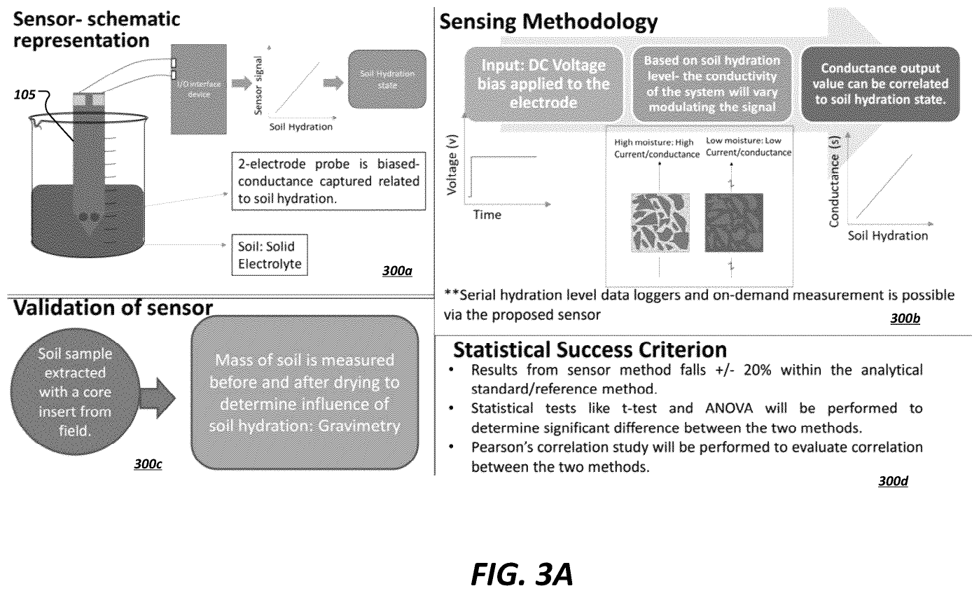

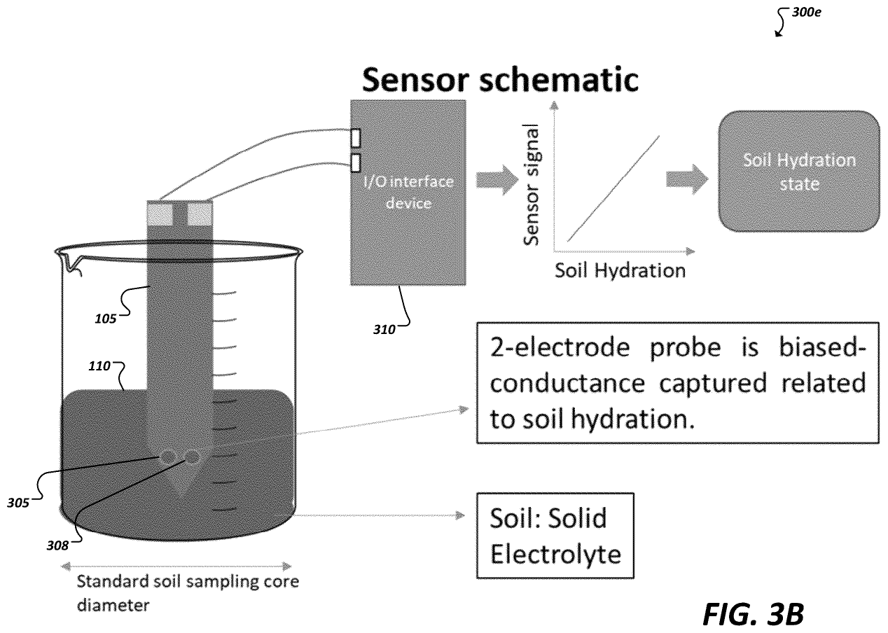

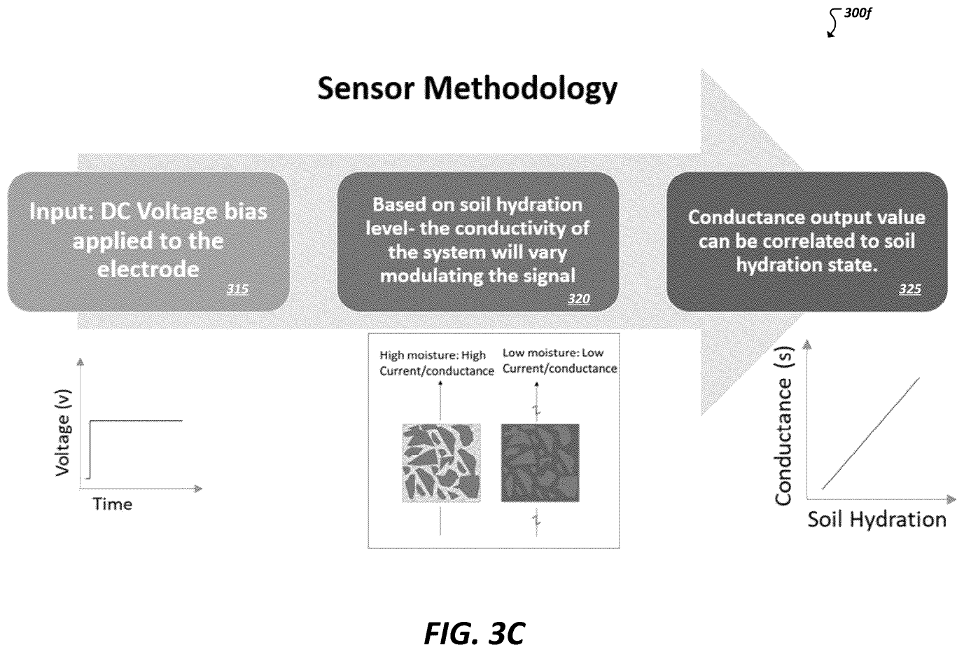

The principles above may be employed to develop a variety of different interfacial soil sensors to measure a variety of different soil health attributes. For instance, FIGS. 3A-3C are diagrams 300a-f illustrating example principles of an example soil hydration state sensor. In one example, a soil hydration state sensor may be implemented as a two-electrode sensor, for instance, fabricated on a printed circuit board (PCB) substrate that records signal as electrode 1 vs electrode 2. FIG. 3A shows an overview of the sensor-schematic representation of such a sensor (at 300a) as well as the overall sensing methodology (at 300b). A soil hydration sensor may measure the moisture content within a soil sample. Validation of the sensor (and training of correlation engine used to interpret signals generated by the sensor) may involve comparing readings of the soil sample against moisture measurements of the sample using alternative means (e.g., gravimetry), with this calibrated alternative sensor readings used as the ground truth for establishing this correlation.

Continuing with the example of a soil hydration state sensor, as shown in FIG. 3B, two electrodes 305, 308 may be provided on the sensor to contact the soil sample 110. In some instances, one or both of the electrodes may be coated with an RTIL layer. In other examples, the sensor 105 may generate a physical/chemiresistive measure and hence no coating may be required and evaluated, in some examples. The electrodes 305, 308 may be coupled with corresponding terminals to couple the sensor 105 to an I/O interface device 310, which may include computational logic to process signals generated at the electrodes and interpret the signals as corresponding to particularly soil hydration level values. As illustrated in FIG. 3C, the methodology of the sensor may include recording a Conductance (1/resistance) and Capacitive output between two electrodes (E1 vs E2) 305, 308 when a direct current (DC) input bias is applied 315. The conductivity measured at the sensor will be expected to vary based on soil hydration level (at 320). These output signals may be correlated 325 and translated against soil hydration levels such that the output signals of the sensor 105 may be processed to derive a soil hydration levels (e.g., as a percentage value), among other example hydration sensor implementations utilizing the principles discussed herein.

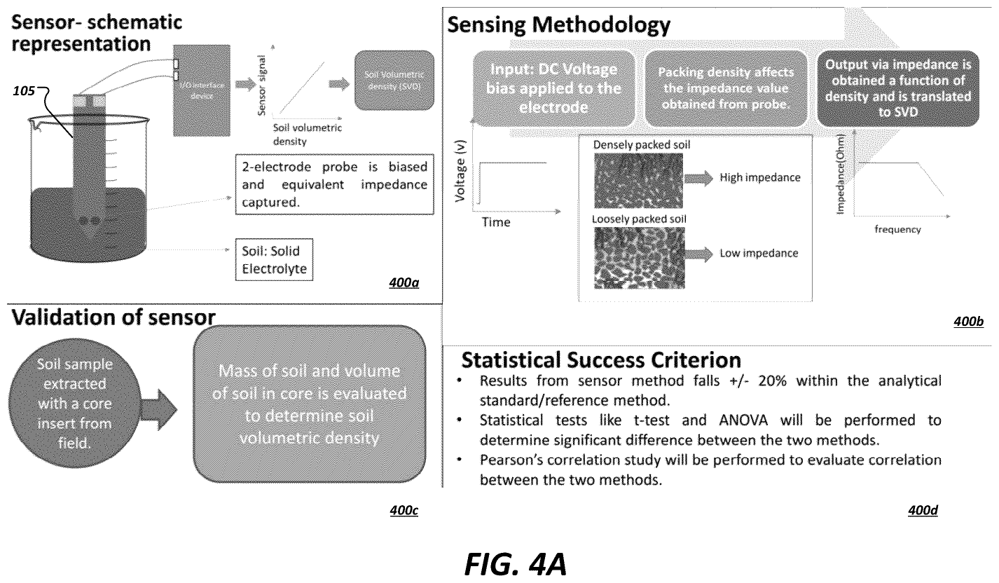

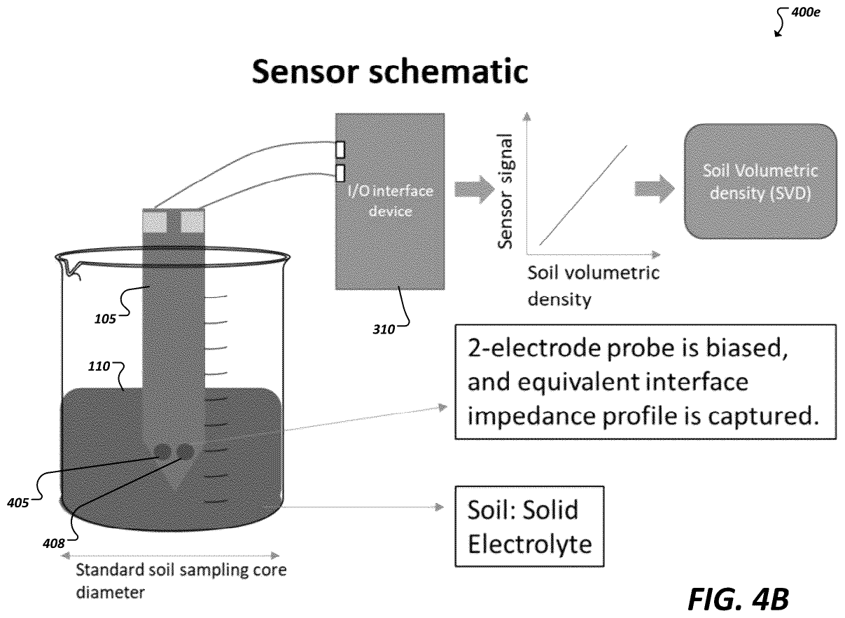

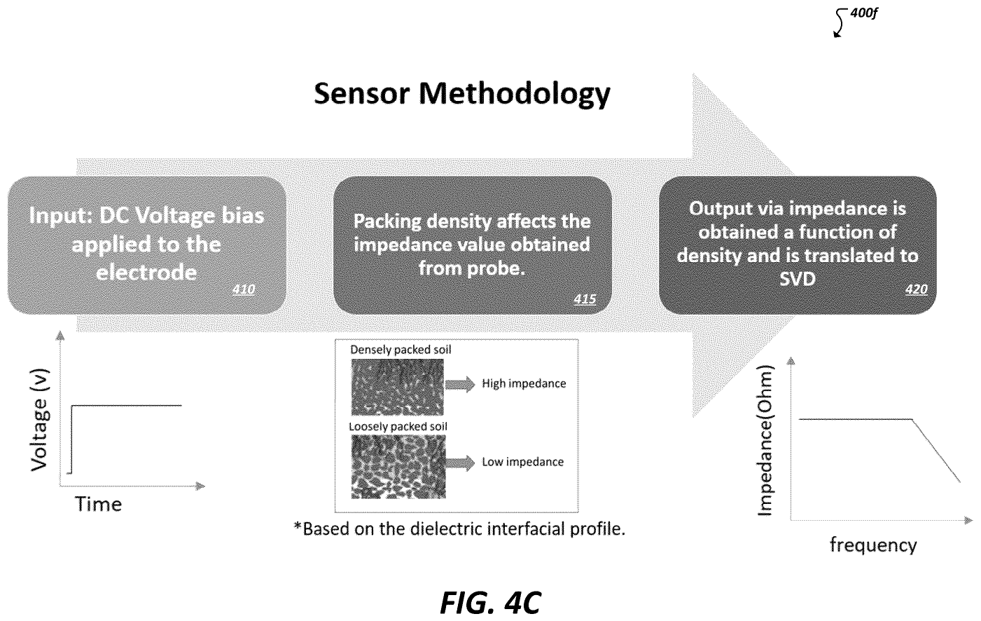

FIGS. 4A-4C are diagrams 400a-f illustrating principles of an example soil volumetric density sensor. In one example, a soil volumetric density sensor may be implemented as a two-electrode sensor fabricated on a printed circuit board (PCB) substrate that probes the sensor-soil (electrolyte) interface and translates to an informative signal. FIG. 4A shows an overview of the sensor-schematic representation of such a sensor (at 400a) as well as the overall sensing methodology (at 400b). A soil volumetric density sensor may measure the moisture content within a soil sample 110. Validation of the sensor 105 (and training of correlation engine used to interpret signals generated by the sensor) may involve comparing readings of the soil sample against volumetric density measurements of the sample using alternative, with this calibrated alternative sensor readings used as the ground truth for establishing this correlation.

Continuing with the example of a soil volumetric density sensor, as shown in FIG. 4B, two electrodes 405, 408 may be provided on the sensor to contact the soil sample 110. In some instances, one or both of the electrodes may be coated with an RTIL layer. In other examples, the sensor 105 may generate a physical/chemiresistive measure and hence no coating may be required and evaluated, in some examples. As in other examples, an I/O interface device may be provided to couple to the sensor 105 to process signals of the electrodes. In other implementations, data processing logic (e.g., a correlation engine) may be collocated on the sensor 105, among other example implementations. As illustrated in FIG. 4C, the methodology of the sensor may include tracking soil bulk composition and thereby soil volumetric density measurement will be based on probing the soil diffuse double layer (DDL) dielectric to evaluate a thorough chemical profile of the soil matrix and can be used to label the interfacial dielectric (RC-resistive and capacitive) parameters towards soil volumetric density in g/cm3. For instance, a DC voltage bias may be applied to the electrodes 410 and impedance measured at the electrodes (at 415) based on the observation that packing density affects the measured impedance value. This impedance output may be correlated and translated into a corresponding soil volumetric density (SVD) measurement (at 420).

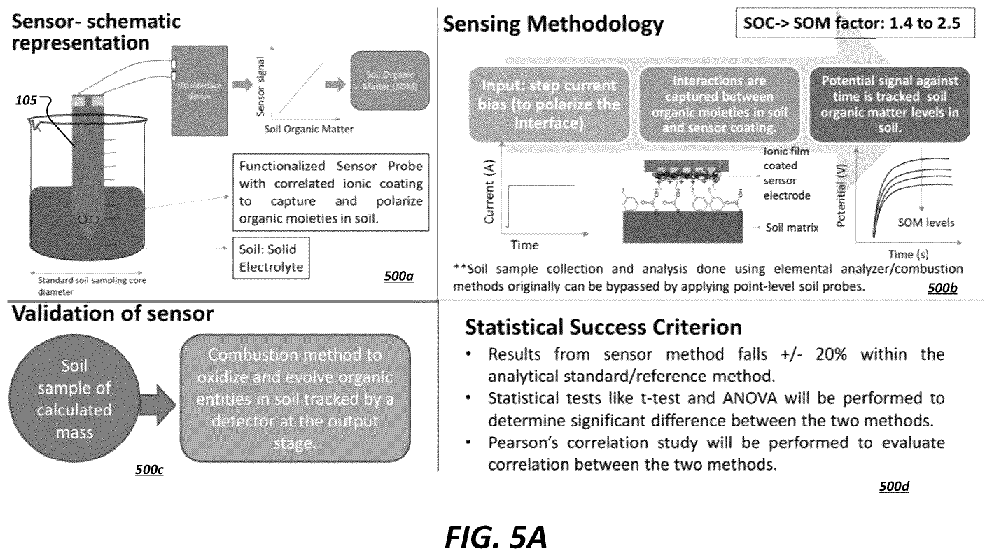

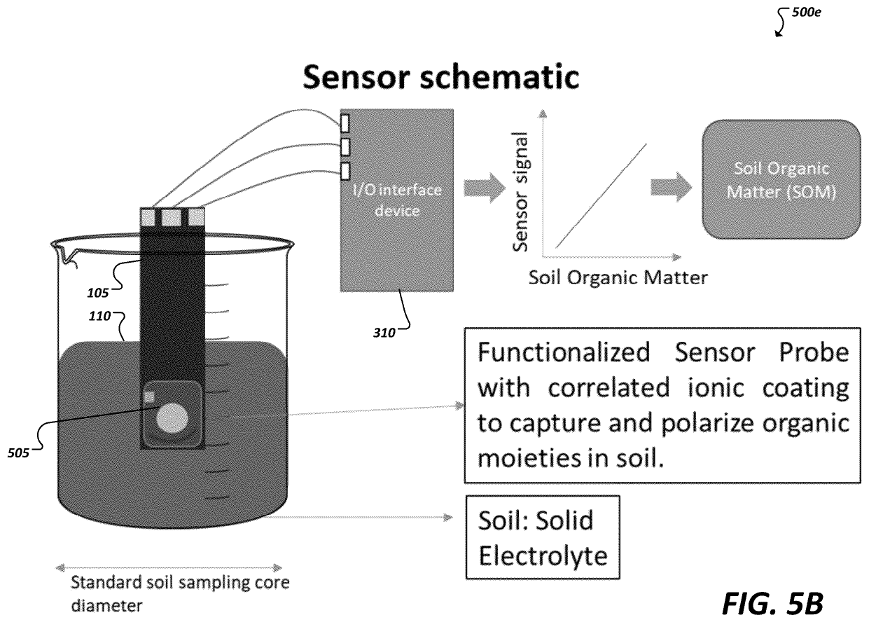

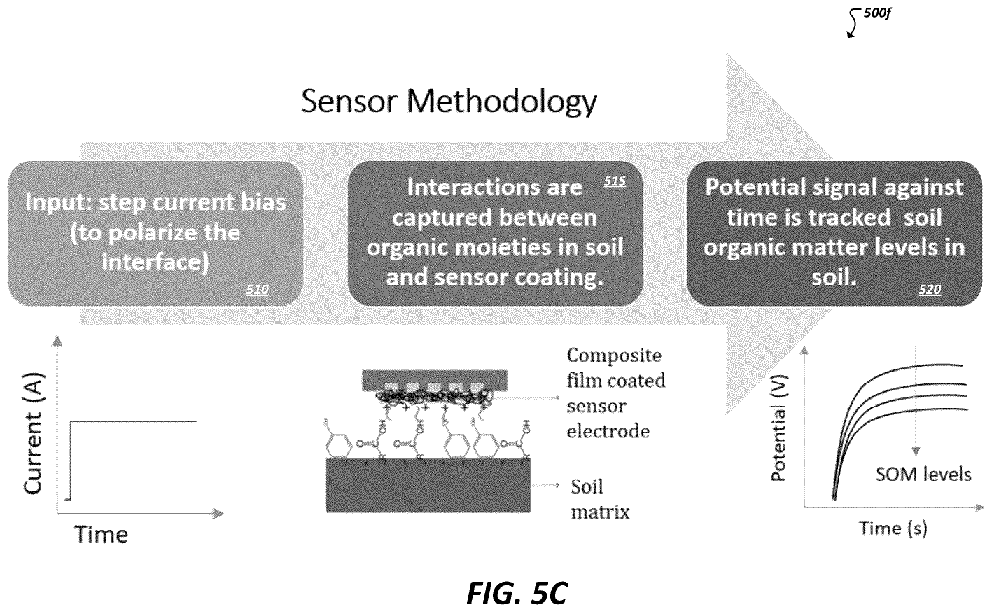

FIGS. 5A-5C are diagrams 500a-f illustrating principles of an example soil organic matter (SOM) sensor. In one example, an SOM sensor may be implemented as a three-electrode sensor fabricated on a PCB substrate that is functionalized by a film coating of organic framework structures for the capture/interaction of organic moieties in the soil with the sensor layer. FIG. 5A shows an overview of the sensor-schematic representation of such a sensor (at 500a) as well as the overall sensing methodology (at 500b). A soil volumetric density sensor may measure the moisture content within a soil sample 110. Validation of the sensor 105 (and training of correlation engine used to interpret signals generated by the sensor) may involve comparing the sensor's readings of the soil sample against SOM measurements of the sample obtained using alternative techniques (e.g., combustion method), with this calibrated alternative sensor readings used as the ground truth for establishing this correlation.

As shown in FIG. 5B, an example SOM sensor may include three electrodes (e.g., at 505) and these electrodes may couple to an I/O interface device 310 via three corresponding terminals. The methodology of the sensor may include dual mode analysis via voltammetric and potentiometric models, to track interaction and capture of target organic functional groups in soil within the sensing area to changes in current/potential as a function of output change in soil organic matter (SOM) levels (e.g., in parts per million (ppm)). In one example, a composite coating may be employed on the SOM sensor including crystalline polymer structures and in particular covalent organic frameworks (COF). This membrane layer is then to be fused with correlated ionic liquid coatings (Imidazolium and Phosphonium based). This composite film can then be either drop-casted, screen printed/spin-coated onto the electrode area 505. The combination of a porous polymer framework membranous layer with zwitter-ionic elements allows the organic moieties (OM) in soil to interact with the sensor layer and these OM levels can be tracked by an example transduction methodology (e.g., as discussed above).

In one example, organic matter present in soil drives the electrochemical charge of the SOM sensor due to presence of the electroactive moieties that are carbon based. Similar to an electrochemical cell comprising of metallic electrodes embedded with the electrolyte, in this case soil is the electrolyte and the RTIL coated metallic electrodes serve as the embedded electrodes to complete the electrochemical cell. As shown in FIG. 5C, a step current bias may be applied 510 at the electrode to polarize the interface. For instance, an interface may be formed between the electrode and electrolyte that is perturbed and polarized by applying a pulsed DC bias to the electrode system and measuring the effect of the polar current as a function of the SOM profile. Interactions may be captured 515 at the electrodes between organic moieties in the soil and sensor coating. The potential signal measured at the electrodes may be tracked against time to derive the SOM levels in the soil. In some examples, the anatomy of the functionalized film may have a higher affinity towards organic carbon pools in soil to make the film a composite ionic film. Soil organic matter can be used as an indirect measure of soil organic carbon (SOC). Such a sensor may be utilized as an in-situ, on-demand, electrochemical soil analysis platform.

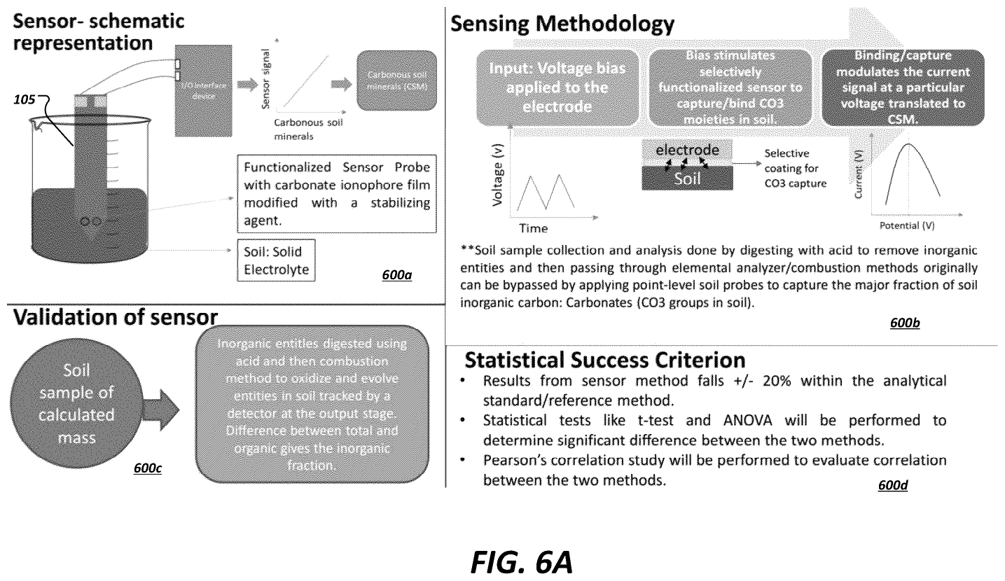

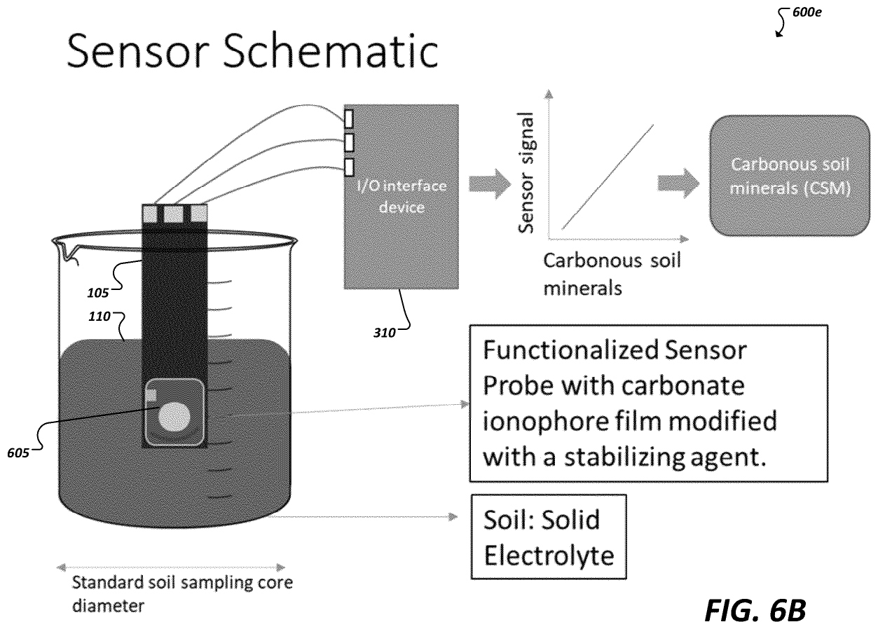

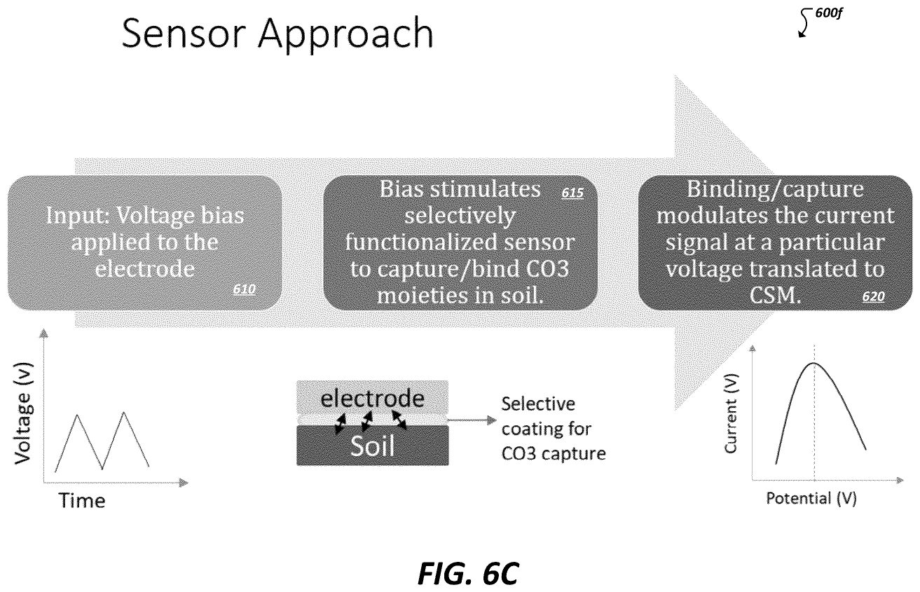

FIGS. 6A-6C are diagrams 600a-f illustrating example principles of an example carbonous soil minerals (CSM) sensor. In one example, a CSM sensor may be implemented as a three-electrode sensor fabricated on a PCB substrate and functionalized by an ionophore dominant membrane coating to selectively capture and interact with carbon-based minerals (carbonates) in the soil sample matrix. FIG. 6A shows an overview of the sensor-schematic representation of such a sensor (at 600a) as well as the overall sensing methodology (at 600b). A CSM sensor may measure the moisture content within a soil sample 110. Validation of the sensor 105 (and training of correlation engine used to interpret signals generated by the sensor) may involve comparing the sensor's readings of the soil sample against CSM measurements of the sample obtained using alternative techniques (e.g., digesting inorganic entities using acid and then combustion method to oxidize evolve entities in soil tracked by a detector at the output stage—the difference between the total and organic gives the inorganic fraction), with this calibrated alternative sensor readings used as the ground truth for establishing this correlation.

Turning to FIG. 6B, an electrode region 605 on the sensor 105 may include three electrodes to generate signals to derive CSM values for the soil sample 110. Methodology of the sensor may include dual mode analysis via Coulometric and Potentiometric modes to track interaction and capture of target carbon-based minerals (carbonates) from inorganic sources in the soil matrix within the sensing area to changes in charge/potential as a function of output change in carbonous soil minerals (CSM) levels (e.g., in ppm). In one example, a functionalized coating of the sensor may include ion-specific receptor elements (Carbonate ionophore based) as the main sensing element. To help dissolve and prepare the ionophore layer, a plasticizer framework (like Dioctyl adipate (DOA), 2-Nitrophenyl octyl ether (NPOE)) is employed. This capture probe layer is then amalgamated into a polymer membrane (like PVC) to create a stable film. This composite may be pieced together by ion sensitive membranes that offer ionic stability-tetradodecylammonium tetrakis (4-chlorophenyl) borate and tridodecylmethylammonium chloride (TDMACI). This aggregate film can then be either drop-casted, screen printed/spin-coated onto the electrode area. The presence of a functional and specific membrane layer that is stable-physically (mechanical stability) and chemically (ionic stability) and holds the ion-specific receptor (ionophore) that selectively captures carbonous minerals in soil is tracked by the signal transduction method utilized by the sensor.

As illustrated in FIG. 6C, a voltage bias may be applied 610 to the electrodes of the CSM sensor, with the bias stimulating 615 the selectively functionalized sensor to capture or bind to CO3 moieties present in the soil sample. This binding/capture causes a modulation 620 of the current signal at a particular voltage. These electrochemical readings may be translated to CSM values for the soil sample (e.g., using a correlation engine).

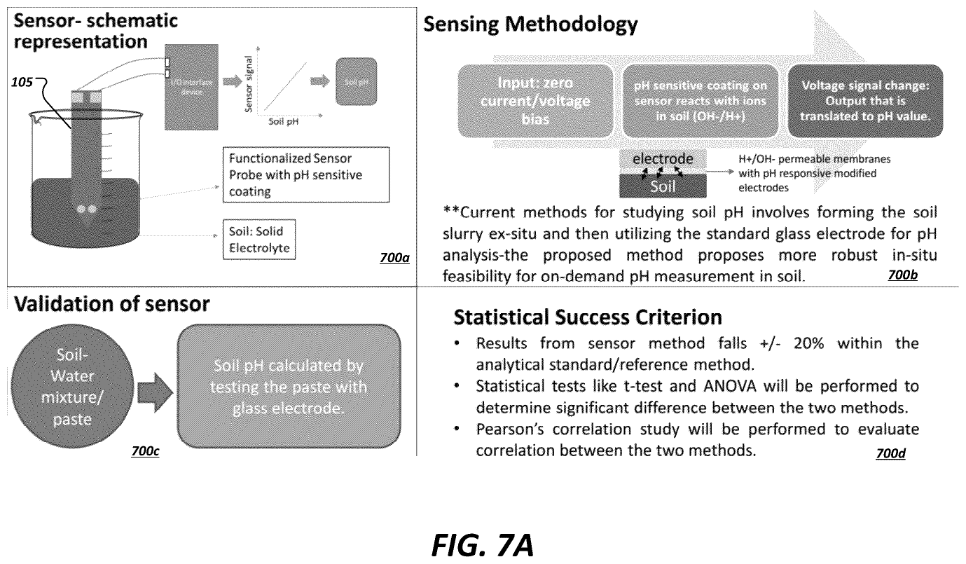

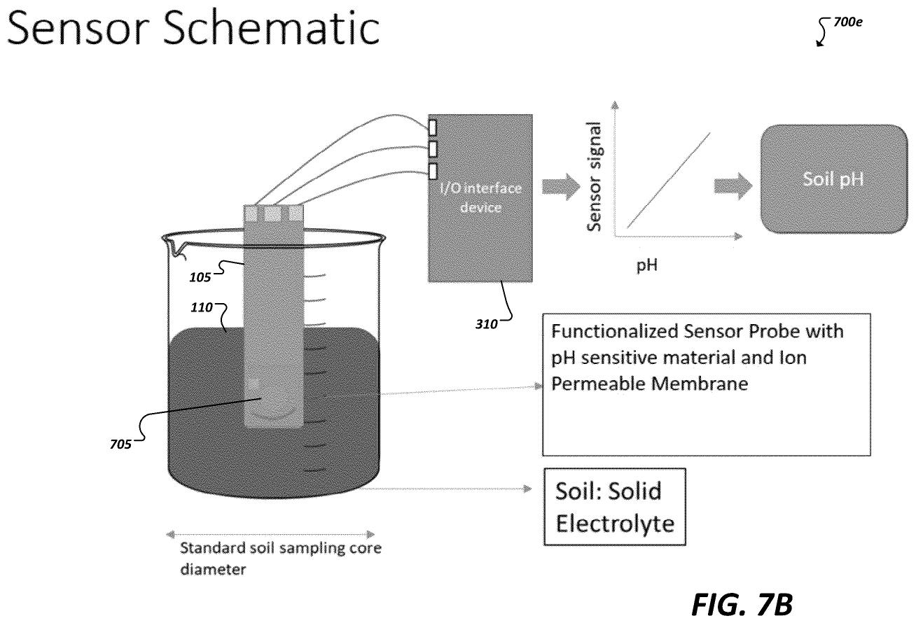

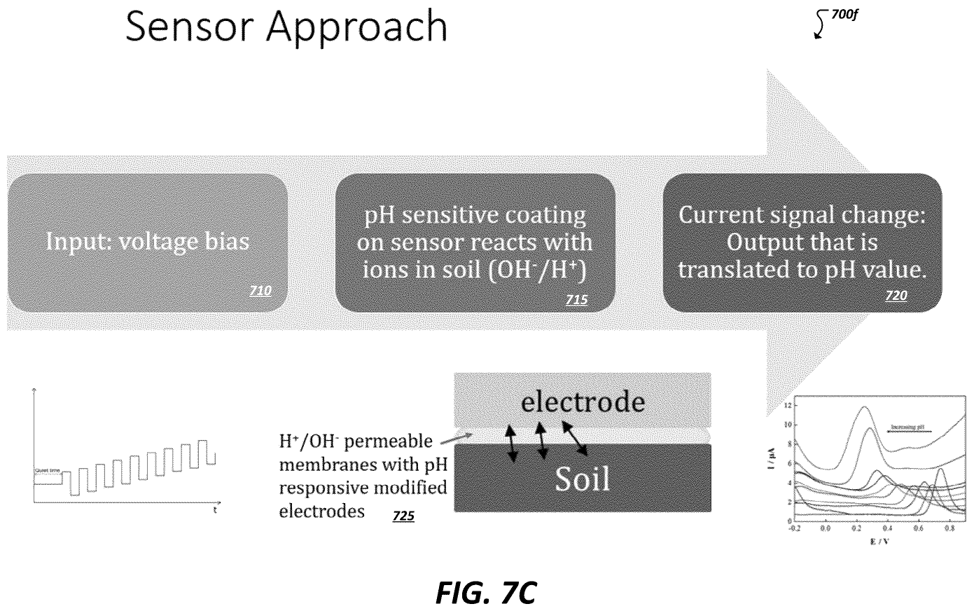

FIGS. 7A-7C are diagrams 700a-f illustrating principles of an example soil pH sensor. In one example, a soil pH sensor may be implemented as a 2- or 3-electrode sensor fabricated on a PCB substrate functionalized by a pH sensitive coating that causes a signal change when ionic species are present in a soil sample. FIG. 7A shows an overview of the sensor-schematic representation of such a sensor (at 700a) as well as the overall sensing methodology (at 700b). A soil pH sensor may measure the moisture content within a soil sample 110. Validation of the sensor 105 (and training of correlation engine used to interpret signals generated by the sensor) may involve comparing the sensor's readings of the soil sample against pH measurements of the sample obtained using alternative techniques (e.g., using a glass electrode), with this calibrated alternative sensor readings used as the ground truth for establishing this correlation.

As shown in FIG. 7B, an example pH soil sensor may include an electrode region 705 that includes two or three electrodes. The electrodes may be functionalized with pH sensitive material and an ion permeable membrane. For instance, the potentiometric analysis may be utilized to track ionic species present in the soil sample, which then modulates the overall output signal due to the presence of the ion sensitive membrane layer and the pH sensitive element. This ionic movement causes an equivalent potential change functional to the amount (concentration) of ionic species in soils represented by pH values (3-9). In some implementations, a soil pH sensor may utilize a coating including a pH sensitive receptor element like (Alizarin & 9,10-phenanthraquinone (PAQ)) that interacts with ionic entities in soil and is functional to pH change. This receptor element may be integrated into an amorphous, conductive layer such as carbon-ink that holds/encapsulates the pH sensitive material within its structure. This composite may be sealed by an ion exchange polymer membrane like Nafion, PMMA, etc. that forms a cross-network holding together the coating on the functional sensor surface. A functional and specific membrane layer that is ion-conductive and protects the functional layer beneath the pH sensitive element may be provided and held together by a conductive carbon layer, which has the ability to modulate charge. This functional element (e.g., Alizarin) interacts with ionic species in soil causing an overall ion exchange activity (reversible) that is tracked by the signal transduction method utilized by the sensor.

As shown in FIG. 7C, methodology of an example pH sensor may include providing 710 a voltage bias to the electrodes enhanced by a pH sensitive coating 725 (e.g., RTIL-based). The pH sensitive coating on the sensor may react with ions in the soil (e.g., OH−/H+) and output signals of the sensor may be modulated based on the presence of such ions. These signals (e.g., changes in signal current) may be correlated with pH values (e.g., using a correlation engine or other logic), among other example features and implementations.

While the examples of FIGS. 3A-7C illustrate example soil sensor implementations, it should be appreciated that these are presented as illustrative examples only and that a variety of other, additional interfacial soil sensors may be implemented based on and applying the principles described herein, including sensors with varying form factors and substrates, sensors applying different ionic layers or coatings, and sensors capable of being used to measure other attributes of soil health. Moreover, multiple sensor designs may be applied and integrated within a single sensor device to enable the device to concurrently measure multiple different soil health attributes for a corresponding soil sample matrix. Indeed, an array of soil health attributes may be advantageously measured using soil sensor systems such as described herein to develop measurements of the overall health of a plot of ground (e.g., farm land, ranch land, orchard plots, vineyards, and the like).

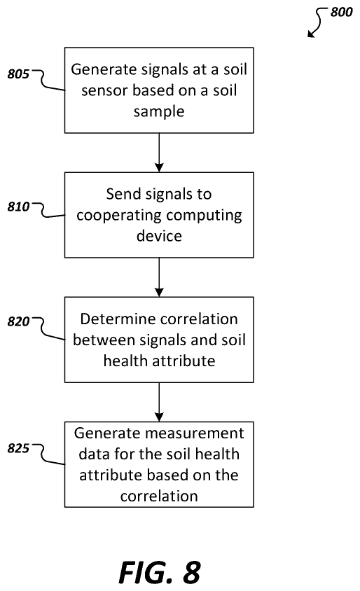

FIG. 8 is a simplified flow diagram 800 illustrating an example technique involving the use of an example in-situ soil sensor. The sensor may be deployed in a particular soil sample (either isolated in a container or representing a portion of a large plot of ground or soil. Electrodes of the soil sensor may be in prolonged and direct contact with the soil and may be configured to react to, measure, or detect electrochemical properties of the soil. The sensor, through the electrodes, may generate signals based on RTIL-based coatings applied to one or more electrodes of the sensor. The signals may be sent to a cooperating computing device, which include computer processing hardware and logic to determine 820 correlations between the generated signals and various soil health attributes related to the electrochemical properties measured by the sensor. In some implementations, the cooperating computing device may be different from and remote from the sensor device. In other implementations, the computing device and its hardware may be integrated with the sensor device. Measurement data may be generated 825 based on the determined correlation to indicate a measure of the corresponding soil health attribute. This information may be further used, stored, shared, or tracked to assess, on a continuing basis, the health of this portion of the soil, and through the deployment of multiple such sensors in multiple nearby soil samples, the overall health of a plot of land and its soil, among other example applications and benefits.

Note that in this document, references to various features (e.g., elements, structures, modules, components, steps, operations, characteristics, etc.) included in “one embodiment”, “example embodiment”, “an embodiment”, “another embodiment”, “some embodiments”, “various embodiments”, “other embodiments”, “alternative embodiment”, and the like are intended to mean that any such features are included in one or more embodiments of the present disclosure, but may or may not necessarily be combined in the same embodiments. Furthermore, the words “optimize,” “optimization,” and related terms are terms of art that refer to improvements in speed and/or efficiency of a specified outcome and do not purport to indicate that a process for achieving the specified outcome has achieved, or is capable of achieving, an “optimal” or perfectly speedy/perfectly efficient state.

In general, computing systems, which interface with a biosensor via a wired or wireless communication channel, can include electronic computing devices operable to receive, transmit, process, store, or manage data and information associated with the biosensor and other subsystems of the computing system. As used in this document, each of the terms “computer,” “processor,” “processor device,” “microcontroller,” or “processing device” is intended to encompass any suitable data processing apparatus. For example, while the microcontroller may be implemented, in some examples, as a single device within the computing system, in other implementations the processing functionality of the system may be implemented using a plurality of computing devices and processors, such as a fog computing system, server pools, a cloud computing system, or other distributed computing system including multiple computers. Further, any, all, or some of the computing devices may be adapted to execute any operating system, including Linux, UNIX, Microsoft Windows, Apple OS, Apple iOS, Google Android, Windows Server, etc., as well as virtual machines adapted to virtualize execution of a particular operating system, including customized and proprietary operating systems.

In some implementations, all or a portion of a computing platform may function as a wearable device, standalone biosensor device, or other sensor device. A sensor device may connect to and communicate with other computing devices through wired or wireless network connections. For instance, wireless network connections may utilize wireless local area networks (WLAN), such as those standardized under IEEE 802.11 family of standards, home-area networks such as those standardized under the Zigbee Alliance, personal-area networks such as those standardized by the Bluetooth Special Interest Group, cellular data networks, such as those standardized by the Third-Generation Partnership Project (3GPP), and other types of networks, having wireless, or wired, connectivity. For example, an endpoint device may also achieve connectivity to a secure domain through a bus interface, such as a universal serial bus (USB)-type connection, a High-Definition Multimedia Interface (HDMI), or the like.

It is also important to note that the operations and steps described with reference to the preceding FIGURES illustrate only some of the possible scenarios that may be executed by, or within, the system. Some of these operations may be deleted or removed where appropriate, or these steps may be modified or changed considerably without departing from the scope of the discussed concepts. In addition, the timing of these operations may be altered considerably and still achieve the results taught in this disclosure. The preceding operational flows have been offered for purposes of example and discussion. Substantial flexibility is provided by the system in that any suitable arrangements, chronologies, configurations, and timing mechanisms may be provided without departing from the teachings of the discussed concepts.

The following examples pertain to embodiments in accordance with this Specification. Example 1 is an apparatus including: a soil sensor including an electrode, where the electrode includes a room temperature ionic liquid (RTIL)-based film layer, where the electrode is to be brought into direct contact with a soil sample, and the soil sensor is functionalized to detect one or more characteristics of the soil sample and generate a signal corresponding to the one or more characteristics.

Example 2 includes the subject matter of example 1, where the electrode is to measure electrochemical features of the soil sample, the one or more characteristics include soil health attributes of the soil sensor, and the one or more characteristics are derivable from the electrochemical features.

Example 3 includes the subject matter of example 2, where the soil sensor is to continuously generate signals corresponding to the electrochemical features measured for the soil sample by the soil sensor.

Example 4 includes the subject matter of any one of examples 1-3, where the RTIL-based film layer serves as an active element for the soil sensor.

Example 5 includes the subject matter of any one of examples 1-3, where the RTIL-based film layer serves a binding element for the soil sensor, and the binding element stabilizes use of another substance on the electrode used as an active element for the soil sensor.

Example 6 includes the subject matter of any one of examples 1-5, where the RTIL-based film layer serves as a support electrolyte for the electrode.

Example 7 includes the subject matter of any one of examples 1-6, where the one or more characteristics include a soil hydration level of the soil sample.

Example 8 includes the subject matter of any one of examples 1-6, where the one or more characteristics include presence of chemical components within the soil sample.

Example 9 includes the subject matter of example 8, where the chemical components include a level of organic compounds within the soil sample.

Example 10 includes the subject matter of example 8, where the chemical components include a level of carbon-based minerals within the soil sample.

Example 11 includes the subject matter of examples 1-6, where the one or more characteristics include volumetric density of the soil sample.

Example 12 includes the subject matter of any one of examples 1-11, where the RTIL film layer includes a 1-butyl-3-methyl imidazolium tetra fluoroborate [BMIM] [BF4] RTIL.

Example 13 is a method including: detecting, at a soil sensor in a soil sample, electrochemical attributes of the soil sample, where the soil sensor includes one or more electrodes in direct contact with the soil, the one or more electrodes include a layer including a room temperature ionic liquid (RTIL)-based film; and generating, at the soil sensor, a signal to identify the electrochemical attributes of the soil sample, where the electrochemical attributes correspond to a soil health attribute of the soil, and the RTIL-based film is used to enable sensing of the soil health attribute.

Example 14 includes the subject matter of example 13, where the soil health attribute includes one of pH, soil hydration level, presence of soil organic matter, soil volumetric density, presence of carbon-based minerals, or presence of another chemical compound.

Example 15 includes the subject matter of any one of examples 13-14, further including: determining a correlation between the signal and measurement of the soil health attribute; and generating an indication of the measurement of the soil health attribute based on the correlation.

Example 16 includes the subject matter of any one of examples 13-15, where the soil sensor is to continuously generate signals corresponding to the electrochemical features measured for the soil sample by the soil sensor.

Example 17 includes the subject matter of any one of examples 13-16, where the RTIL-based film serves as an active element for the soil sensor.

Example 18 includes the subject matter of any one of examples 13-16, where the RTIL-based film serves a binding element for the soil sensor, and the binding element stabilizes use of another substance on the electrode used as an active element for the soil sensor.

Example 19 includes the subject matter of any one of examples 13-16, where the RTIL-based film serves as a support electrolyte for the electrode.

Example 20 includes the subject matter of any one of examples 13-19, where the RTILfilm layer includes a 1-butyl-3-methyl imidazolium tetra fluoroborate [BMIM] [BF4] RTIL.

Example 21 is a system including means to perform the method of any one of examples 13-20.

Example 22 is a system including: a set of interfacial soil sensors, where at least one sensor in the set of interfacial soil sensors includes an electrode with a room temperature ionic liquid (RTIL)-based film, and the sensor is to generate signals corresponding to detection of one or more soil health attributes of a corresponding soil sample, where the RTIL-based film is used to enable sensing of the one or more soil health attributes.

Example 23 includes the subject matter of example 22, where the set of interfacial soil sensors includes a plurality of interfacial soil sensors, and each of the plurality of interfacial soil sensors uses a respective RTIL-based film to enable sensing of a corresponding soil health attribute.

Example 24 includes the subject matter of example 23, where the plurality of interfacial soil sensors are deployed in a respective portion of a plot of soil and measure soil health attributes of soil samples corresponding to the respective portion of the plot.

Example 25 includes the subject matter of example 24, further including one or more gateway devices to receive sensor data generated by the plurality of interfacial soil sensors over a wireless network.

Example 26 includes the subject matter of any one of examples 22-25, where the at least one sensor utilizes the RTIL-based film as at least one of an active element, a support electrolyte, or a binding element for the electrode.

Example 27 includes the subject matter of any one of examples 22-26, where the one or more soil health attributes include a soil hydration level of the soil sample.

Example 28 includes the subject matter of any one of examples 22-26, where the one or more soil health attributes include presence of chemical components within the soil sample.

Example 29 includes the subject matter of example 28, where the chemical components include a level of organic compounds within the soil sample.

Example 30 includes the subject matter of example 28, where the chemical components include a level of carbon-based minerals within the soil sample.

Example 31 includes the subject matter of any one of examples 22-26, where the one or more soil health attributes include volumetric density of the soil sample.

Example 32 includes the subject matter of any one of examples 22-26, where the one or more soil health attributes include soil pH.

Example 33 includes the subject matter of any one of examples 22-32, where the RTILfilm layer includes a 1-butyl-3-methyl imidazolium tetra fluoroborate [BMIM] [BF4] RTIL.

Example 34 includes the subject matter of any one of examples 22-33, where the soil sensor is to continuously generate signals corresponding to detection of one or more soil health attributes.

Example 35 includes the subject matter of any one of example 22-35, further including: a data processor; and a correlation engine executable by the data processor to: access data describing the signals generated by the at least one sensor; determine a correlation between the signals and the corresponding soil health attributes; and determine a measurement of an amount of the soil health attributes in the soil sample based on the correlation.

Example 36 is a sensor device including: a soil hydration state sensor including: two or more electrodes; circuitry to: detect an output signal including one or more of conductance and capacity between the two or more electrodes when a direct current input bias is applied; and translate the output signal into a soil hydration level value.

Example 37 is a sensor device including: a soil volumetric density sensor including: two or more electrodes; circuitry to: probe a soil diffuse double layer (DDL) dielectric; and determine a soil volumetric density value.

Example 38 is a sensor device including: a soil organic matter sensor including: at least three electrodes, where at least a particular one of the at least three electrodes is functionalized by a film coating of organic framework structure to correspond to organic matter in soil; and circuitry to: detect changes in current or potential; and determine an amount of organic matter in the soil based on the changes.

Example 39 includes the subject matter of example 38, where the film coating includes a room temperature ionic liquid (RTIL)-based film coating.

Example 40 includes the subject matter of example 39, where the RTIL-based film coating includes a BMIM BF4 RTIL.

Example 41 includes the subject matter of example 39, where the RTIL-based film coating includes a tetra fluro burate anion-based RTIL.

Example 42 is a sensor device including: a carbonous soil minerals sensor including: at least three electrodes, where at least a particular one of the at least three electrodes is functionalized by an ionophore dominant membrane coating to selectively capture and interact with carbon-based minerals in soil; and circuitry to: detect changes in current or potential; and determine an amount of carbon-based minerals (carbonates) in the soil based on the changes.

Example 43 is a sensor device including: a pH sensor including: at least two electrodes, where at least one of the at least two electrodes is functionalized by a pH sensitive coating that causes a signal change when ionic species are present in soil; circuitry to: detect a potential change at the at least two electrodes; and determine a pH value associated with the soil based on the potential change.

Example 44 is a system including at least one of the sensor devices of examples 36-43.

Example 45 includes the subject matter of example 44, including two or more of the sensor devices of examples 36-43.

Although the present disclosure has been described in detail with reference to particular arrangements and configurations, these example configurations and arrangements may be changed significantly without departing from the scope of the present disclosure.

Numerous other changes, substitutions, variations, alterations, and modifications may be ascertained to one skilled in the art and it is intended that the present disclosure encompass all such changes, substitutions, variations, alterations, and modifications as falling within the scope of the appended claims.

1.-33. (canceled)

34. An apparatus comprising:
a soil sensor comprising an electrode, wherein the electrode comprises a room temperature ionic liquid (RTIL)-based film layer,
wherein the electrode is to be brought into direct contact with a soil sample, and the soil sensor is functionalized to detect one or more characteristics of the soil sample and generate a signal corresponding to the one or more characteristics.

35. The apparatus of claim 34, wherein the electrode is to measure electrochemical features of the soil sample, the one or more characteristics comprise soil health attributes of the soil sensor, and the one or more characteristics are derivable from the electrochemical features.

36. The apparatus of claim 35, wherein the soil sensor is to continuously generate signals corresponding to the electrochemical features measured for the soil sample by the soil sensor.

37. The apparatus of claim 34, wherein the RTIL-based film layer serves as an active element for the soil sensor.

38. The apparatus of claim 34, wherein the RTIL-based film layer serves a binding element for the soil sensor, and the binding element stabilizes use of another substance on the electrode used as an active element for the soil sensor.

39. The apparatus of claim 34, wherein the RTIL-based film layer serves as a support electrolyte for the electrode.

40. The apparatus of claim 34, wherein the one or more characteristics comprise a soil hydration level of the soil sample.

41. The apparatus of claim 34, wherein the one or more characteristics comprise presence of chemical components within the soil sample.

42. The apparatus of claim 41, wherein the chemical components comprise one of a level of organic compounds within the soil sample or a level of carbon-based minerals within the soil sample.

43. The apparatus of claim 34, wherein the one or more characteristics comprise volumetric density of the soil sample.

44. The apparatus of claim 34, wherein the RTIL film layer comprises a 1-butyl-3-methyl imidazolium tetra fluoroborate [BMIM] [BF4] RTIL.

45. A method comprising:
detecting, at a soil sensor in a soil sample, electrochemical attributes of the soil sample, wherein the soil sensor comprises one or more electrodes in direct contact with the soil, the one or more electrodes comprise a layer comprising a room temperature ionic liquid (RTIL)-based film; and
generating, at the soil sensor, a signal to identify the electrochemical attributes of the soil sample, wherein the electrochemical attributes correspond to a soil health attribute of the soil, and the RTIL-based film is used to enable sensing of the soil health attribute.

46. The method of claim 45, wherein the soil health attribute comprises one of pH, soil hydration level, presence of soil organic matter, soil volumetric density, presence of carbon-based minerals, or presence of another chemical compound.

47. The method of claim 45, further comprising:
determining a correlation between the signal and measurement of the soil health attribute; and
generating an indication of the measurement of the soil health attribute based on the correlation.

48. The method of claim 45, further comprising generating, at a second soil sensor with one or more electrodes coated with a second RTIL-based film, a second signal, wherein the one or more electrodes of the second soil sensor are in direct contact with the soil, the second signal identifies second electrochemical attributes of the soil sample, wherein the second electrochemical attributes correspond to a different, second soil health attribute of the soil, and the second RTIL-based film is used to enable sensing of the second soil health attribute.

49. A system comprising:
a set of interfacial soil sensors, wherein at least one sensor in the set of interfacial soil sensors comprises an electrode with a room temperature ionic liquid (RTIL)-based film, and the sensor is to generate signals corresponding to detection of one or more soil health attributes of a corresponding soil sample,
wherein the RTIL-based film is used to enable sensing of the one or more soil health attributes.

50. The system of claim 49, wherein the set of interfacial soil sensors comprises a plurality of interfacial soil sensors, and each of the plurality of interfacial soil sensors uses a respective RTIL-based film to enable sensing of a corresponding soil health attribute.

51. The system of claim 49, wherein the at least one sensor utilizes the RTIL-based film as at least one of an active element, a support electrolyte, or a binding element for the electrode.

52. The system of claim 49, further comprising:
a data processor; and
a correlation engine executable by the data processor to:
access data describing the signals generated by the at least one sensor;
determine a correlation between the signals and the corresponding soil health attributes; and
determine a measurement of an amount of the soil health attributes in the soil sample based on the correlation.

53. The system of claim 49, wherein the soil health attribute comprises one of pH, soil hydration level, presence of soil organic matter, soil volumetric density, presence of carbon-based minerals, or presence of another chemical compound.
